(19) United States
(12) Patent Application Publication
(10) Pub. No.: US 2026/0174482 A1
(43) Pub. Date: Jun. 25, 2026

(54) OSTEOSYNTHESIS PLATE

(71) Applicant: Karl Leibinger Asset Management GmbH & Co. KG, Mühlheim an der Donau (DE)

(72) Inventors: Ralf Leibinger, Mühlheim (DE); Frank Reinauer, Emmingen-Liptingen (DE); Steffen Aberle, Königsfeld (DE)

(21) Appl. No.: 19/424,823

(22) Filed: Dec. 18, 2025

(30) Foreign Application Priority Data
Dec. 19, 2024 (DE) 102024138941.9

(51) Int. Cl. A61B 17/80

(52) U.S. Cl. CPC A61B 17/8071

(57) ABSTRACT

An osteosynthesis plate (OP, OP′, OP″, OP′″) for fixation of bone fragments (K1, K2) includes a plate body (P, P′) with at least one bridge section (BU, BU1, BU2) for bridging a gap (S) between the bone fragments (K1, K2). The plate body (P, P′) includes fixation sections (BA1, BA2, BA1′, BA2′, BA3′), each of which is associated with at least one fixation point (BP1, BP2, BP1′, BP2′, BP3′) that is formed on an island section (IA, IA′) connected to the respective associated fixation section (BA2, BA1′, BA2′) exclusively via spring segments (FS1, FS2, FS1′, FS2) that permit relative displacement of the respective fixation section (BA2, BA1′, BA2′) and of the respective island section (IA, IA′) in an elastically resilient manner. At least one of the spring segments (FS1, FS2, FS1′, FS2) is aligned such that, when a load (B) is applied in a main loading direction of the bone (K), a relative displacement takes place between the island section (IA, IA′) and the fixation section (BA2, BA1′, BA2′) associated with this island section (IA, IA′), during which a distance between the island section (IA, IA′) and the at least one bridge section (BU, BU1) increases.

### CROSS-REFERENCE TO RELATED APPLICATION

The present application is related and has right of priority to German Patent Application No. DE102024138941.9 filed on Dec. 19, 2024, which is incorporated by reference in its entirety for all purposes.

### TECHNICAL FIELD

The invention relates generally to an osteosynthesis plate provided for a fixation of bone fragments for the restoration of a bone, in particular a mandible.

### BACKGROUND

In osteosynthesis, two or more bone fragments are connected to one another within the framework of a surgical procedure in order to enable the bone fragments to grow together and thus achieve a restoration of the bone. The goal of osteosynthesis is to stabilize the bone fragments with respect to one another, wherein this stabilization is to take place in a correct position and thus, optionally, while correcting malalignments. In addition to a fixation using wire or screws, osteosynthesis plates are also used depending on the area of application, wherein the respective osteosynthesis plate is placed on the respective bone in the region of a respective gap between the bone fragments and fixated to each of the bone fragments to be connected to one another.

In addition to stabilizing bone fragments after a bone fracture, osteosynthesis plates are also used in part when a bone segment resected elsewhere is to be used at an existing defect in a bone and stabilized for a restoration of the bone. Such a defect can arise, for example, due to the need to remove a section of bone after a tumor disease.

With respect to a healing process of the bone, it has proven advantageous when the bone fragments of the bone to be restored are not rigidly connected to one another via the osteosynthesis plate, but rather when micromotions of the bone fragments relative to one another are made possible via the osteosynthesis plate. In order to enable these micromotions, the fixation points are connected via spring segments to at least one of the bone fragments to be connected.

WO 2023/057477 A1 describes a plate implant for bridging a fracture gap, which plate implant has a longitudinal axis in a direction of the greatest extent of the plate implant and a number of bores mutually spaced apart in the direction of the longitudinal axis. An elastic longitudinal extender element is present, which is provided between at least two bores. The longitudinal extender element is designed such that the plate implant has an increased extensibility in the direction of the longitudinal axis and at the same time high flexural strength and torsional rigidity. As a result, a certain degree of longitudinal movement in the fracture gap is to be enabled, in order to stimulate the bone healing.

### BRIEF SUMMARY

Example aspects of the present invention provide an osteosynthesis plate, during the use of which for the restoration of a bone, a healing process of the bone is further improved.

According to example aspects of the invention, an osteosynthesis plate includes a plate body having a top side and an underside with which the plate body is to be placed on the bone fragments. The plate body has at least one bridge section, which is used to bridge an associated gap extending between the bone fragments. Furthermore, the plate body is equipped with fixation sections located on each side of the at least one bridge section, at which fixation sections, on the underside thereof, the osteosynthesis plate is to be fixated to the bone fragments and with each of which fixation section at least one fixation point is associated for this purpose. The at least one fixation point, on at least one of the fixation sections, is formed on an island section which is connected to the respective associated fixation section of the plate body exclusively via spring segments which permit a relative displacement of the respective fixation section and of the respective island section relative to one another in an elastically resilient manner.

The osteosynthesis plate according to example aspects of the invention is provided for a fixation of bone fragments for the restoration of a bone. This restoration of the bone can be present within the framework of the invention in such a way that bone fragments that have formed due to a fracture of the bone are fixated to one another via the osteosynthesis plate according to example aspects of the invention. In this case, the bone fragments associated with the bone to be restored are therefore fixated and stabilized with respect to one another via the osteosynthesis plate. A restoration of a bone can also be considered within the meaning of the invention to mean, however, that a defect of a bone is closed with a bone segment which has been resected from another bone for this purpose. Accordingly, the bone in this case is restored from its own bone fragments and a bone fragment associated with another bone. The osteosynthesis plate according to example aspects of the invention is then used in this case for the fixation of the bone segment closing the defect with at least one of the bone segments located on each side. Most particularly preferably, the osteosynthesis plate is provided for use in the region of the lower jaw bone (mandible), wherein a restoration can then be achieved either within the framework of a bone fracture or within the framework of closing a bone defect.

The osteosynthesis plate is equipped with a plate body, which is preferably elongate, i.e., extends in particular at least predominantly in a longitudinal direction. The plate body can extend along a longitudinal axis extending in this longitudinal direction or can be designed extending along a trajectory oriented in this longitudinal direction.

The plate body has a top side and an underside, which are oriented, in particular, facing away from one another on the plate body. When the osteosynthesis plate according to example aspects of the invention is used, the plate body is placed on the bone fragments with the underside of the plate body. During this placement, at least one gap, which extends between the bone fragments to be fixated, is bridged by the plate body. For this respective bridging, the plate body is equipped with a respective bridge section, wherein fixation sections of the plate body are located on each side of this bridge section. At these fixation sections, when the osteosynthesis plate is used, a respective fixation to one of the bone fragments separated via the at least one gap is implemented, for the purpose of which at least one fixation point is associated with each of the fixation sections. Preferably, the respective fixation point is formed by a respective through-hole, through which an associated bone screw can be guided. The fixation to the respective bone fragment is implemented by the associated bone screw.

On at least one of the fixation sections of the plate body, the at least one provided fixation point is formed on an island section, which is connected to the associated fixation section exclusively via spring segments, which couple the respective island section to the associated fixation section. These spring segments permit a relative displacement of the island section relative to the associated fixation section in an elastically resilient manner, which means, within the meaning of the invention, that the spring segment elastically deforms during a relative displacement that the island section and the associated fixation section undergo relative to one another when loaded and, when unloaded, returns to its original shape while moving back into an initial position. Preferably, the spring segment is designed as a spring leg extending at least largely linearly.

Preferably, the spring segments permit the relative displacement between island section and fixation section only largely parallel to the top side and the underside and/or in the longitudinal direction. This is the case because, since the gap is also bridged in this direction due to the course of the plate body, relative displacements oriented in this direction result in changes in the gap size, which has proven advantageous with respect to the healing process of the bone to be restored.

Particularly preferably, the respective island section protrudes from the underside of the plate body relative to the associated fixation section, whereby, when the fixation section is fixated to the respective bone fragment, the respective island section rests on the bone fragment and, at the same time, there is open space between the bone fragment and the associated fixation section. As a result, it can be ensured that the relative displacements between island section and fixation section are not impeded by friction due to the fixation section also resting on the bone fragment which is also to be displaced relative to the fixation section.

The osteosynthesis plate is preferably manufactured by an additive manufacturing process, preferably by a 3D-printing process. The osteosynthesis plate preferably consists of titanium, a titanium alloy, of stainless steel, or of a suitable polymer, for example polyether ether ketone (PEEK).

Example aspects of the invention provide at least one of the spring segments that is aligned in such a way that, when a load is applied in a main loading direction of the bone, a relative displacement takes place between the island section and the fixation section associated with this island section, during which a distance between the island section and the at least one bridge section increases.

In other words, at the respective island section, at least one of the associated spring segments is situated in such a way that, when the bone is loaded in a main loading direction, a relative displacement of the respective island section relative to the respective fixation section takes place, during which the respective island section moves away from the respective bridge section.

Such a design of an osteosynthesis plate has the advantage that, when the osteosynthesis plate is used and the bone is loaded in the main loading direction, the gap bridged by the bridge section between the bone segments also enlarges due to the increase in the distance between the respective island section and the bridge section. This is the case because the relative displacement inducing the increase in the distance also results in a relative displacement of the bone segments connected to one another via the osteosynthesis plate, which is accompanied by a widening of the intermediate gap. Due to this enlargement of the gap, it can be ruled out that the gap between the bone segments becomes zero and the bone segments impact one another, which would complicate the healing process of the bone to be restored. When a resected bone segment is inserted into a defect of the bone to be restored, a gap between the bone segments that is too small or non-existent can also thereby be prevented when the resected bone segment is configured with a transition fit with respect to the defect. In general, when the osteosynthesis plate according to example aspects of the invention is used, micromotions between the bone fragments are made possible, whereby the healing process of the bone to be restored is stimulated.

According to example aspects of the invention, at least one of the spring segments on the island section is oriented in such a way that the gap to be bridged with the bridge section enlarges when a load is applied on the bone in the main loading direction. This is achieved by inducing a correspondingly oriented relative displacement of the island section relative to the associated fixation section.

Within the meaning of the invention, “loaded in the main loading direction” of the bone is understood to mean, in particular, an action of force on the bone when same is loaded as usual. Preferably, this loading takes place in such a way that a resulting transverse force is induced on the associated fixation point on the island section. In the case of the preferred example embodiment of the osteosynthesis plate for the fixation of bone fragments for the restoration of a mandible, the load in the main loading direction is, in particular, a biting force to be supported via the mandible.

According to one possible example embodiment of the invention, the at least one spring segment is oriented so as to enlarge the distance in such a way that this at least one spring segment extends at an acute angle to a respective force vector which represents a transverse force that results on the respective fixation point of the associated island section during the application of the load on the bone acting transversely to the associated fixation section. In this process, when the transverse force is introduced, the at least one spring segment induces a conversion of the transverse force into a longitudinal force component which brings about a relative displacement of the island section relative to the respective fixation section directed away from the associated bridge section.

According to a further possible example embodiment of the invention, the island section is connected to the respective fixation section via two respective spring segments. Preferably, the island section and the two associated spring segments are formed by two U-shaped cutouts, the openings of which face one another and which are nested inside one another in the respective fixation section. Advantageously, this enables a one-piece design of the island section and of the associated spring segments with the plate body to be obtained, wherein suitable displacements of the island section relative to the respective fixation section are achievable. The U-shaped cutouts can be designed having the same course or different courses. If the plate body of the osteosynthesis plate is manufactured by an additive manufacturing process, the through-openings may also have been formed in this additive manufacturing process. Alternatively, the through-openings can also be defined in an abrasive or material-removing manner, however.

In a further possible example embodiment of the invention, the spring segments connect the respective island section to the plate body extending symmetrically to the respective island section. Most particularly preferably, the associated spring segments extend with point symmetry to the respective fixation point of the respective island section. In particular in combination with the example variant of the invention in which the spring segment increasing the distance extends at an acute angle to the force vector, the two spring segments extending with point symmetry therefore ensure that force is converted in a manner that increases the distance.

Alternatively or, in the case of a plurality of island sections, also in addition to the aforementioned example embodiment, the associated spring segments can connect the respective island section to the plate body extending asymmetrically to the respective island section. As a result, adjacent island sections on the respective fixation section can be placed closer to one another and/or a fixation point provided on each end can be located closer to the respective end of the fixation section, whereby the number of fixation points on the fixation section can be increased. In a development of the aforementioned example variant, one spring segment of the associated spring segments, when in an unloaded state, can extend at least approximately transversely to the respective fixation section.

In a combination with the example variant of the invention in which the spring segment increasing the distance extends at an acute angle to the force vector, it would also be conceivable within the framework of example aspects of the invention that the associated spring segments extend at differing acute angles to the respective force vector and diverge towards the at least one associated bridge section.

Preferably, a plurality of fixation points is associated with the respective fixation section, wherein each of the fixation points is formed on a respective island section. In particular, in the case of a plurality of or all island sections of the plurality of fixation points, at least one of the associated spring segments is aligned in such a way that the distance between the island section and the associated bridge section increases during the application of the load. As a result, the enlargement of the gap can be brought about via the plurality of island sections together. Particularly preferably, the island sections are formed in a symmetrical arrangement on the respective fixation section, i.e., the respective island sections are formed on the respective fixation section in an identical manner in terms of their alignment and design.

According to a further possible example embodiment of the invention, of the fixation sections located on each side of the at least one bridge section, the at least one fixation point on the one fixation section is formed on the respective island section, whereas the respective at least one fixation point on the other fixation section is fixed in place on the other fixation section. As a result, the fixation point(s) on the one fixation section is/are displaceable relative to this fixation section, whereas the fixation point(s) on the other fixation section is/are rigid.

Within the framework of example aspects of the invention, the fixation points can also be formed on island sections on each side of the at least one bridge section. Accordingly, a relative displaceability of the fixation points is then provided on both fixation sections. In a development of this example embodiment, at least one of the respective associated spring segments on the island sections of the fixation sections arranged on each side of the bridge section is aligned so as to increase the distance during the application of the load. Therefore, when a load is applied in the region of both fixation sections, an enlargement of the gap located between the bone fragments is brought about.

In a further example variant of the invention, the plate body has exactly one bridge section. In this case, the plate body of the osteosynthesis plate is therefore designed to fixate two or more bone fragments to one another and thereby bridge exactly one gap. Alternatively, the plate body can have a plurality of bridge sections for bridging a plurality of gaps, wherein fixation sections are provided on each side of the respective bridge section. In this case, the osteosynthesis plate preferably has exactly two or more bridge sections and, consequently, three or more fixation sections, whereby three or more bone fragments can then be fixated by the osteosynthesis plate for the restoration of the bone.

### BRIEF DESCRIPTION OF THE DRAWINGS

Advantageous embodiments of the invention, which are explained in the following, are shown in the drawings. In the drawings:

FIGS. 1A and 1B show schematic representations of a bone with an osteosynthesis plate according to one example embodiment of the invention fixated thereto, shown in different states,

FIGS. 2A and 2B show schematic views of a bone with an osteosynthesis plate according to a further possible example embodiment of the invention fixated thereto, shown in different states,

FIG. 3 shows a schematic view of a part of an osteosynthesis plate according to a further example embodiment of the invention, and

FIG. 4 shows a perspective representation of an osteosynthesis plate according to a further possible example embodiment of the invention.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

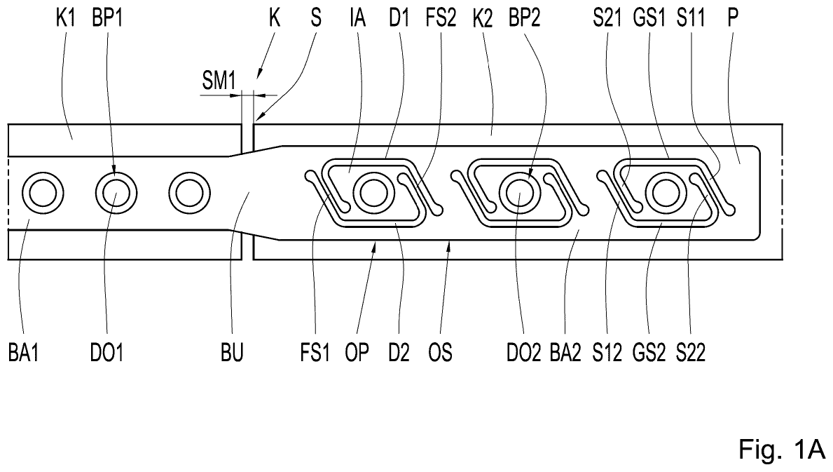

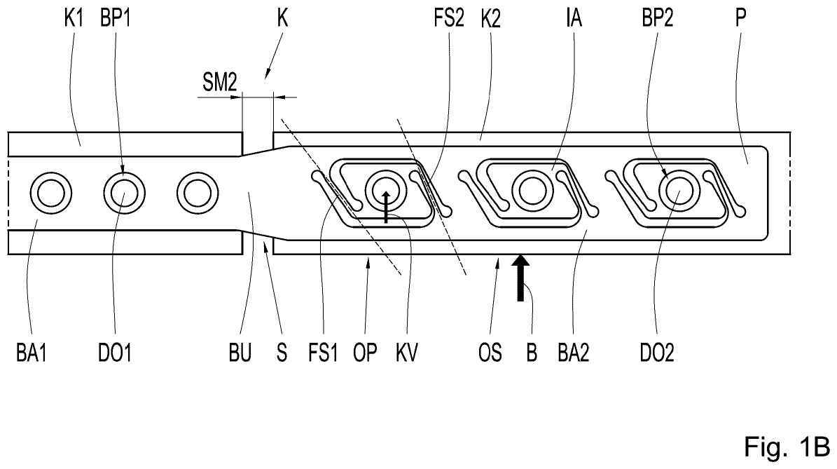

FIGS. 1A and 1B show schematic representations of a bone K with an osteosynthesis plate OP fixated thereto, wherein the bone K is preferably a mandible in this case. The osteosynthesis plate OP is to be fixated to bone fragments K1 and K2 in order to stabilize the bone fragments K1 and K2 relative to one another and thereby enable the bone fragments K1 and K2 to grow together for the restoration of the bone K.

In the present case, the bone fragments K1 and K2 may have formed due to a fracture of the bone K, wherein the osteosynthesis plate OP is then provided for healing this bone fracture by stabilizing the bone fragments K1 and K2 relative to one another. Alternatively, one of the bone fragments K1 and K2 may have been used to close a defect of the bone K, for example, because a part of the bone K had to be removed at this point due to a tumor disease. The bone fragment K1 or K2 is a section of bone resected from another bone, for example, from the fibula.

The osteosynthesis plate OP has an elongate plate body P, which preferably is formed of titanium or a titanium alloy and has been produced, in particular, in an additive manufacturing process, preferably in the 3D printing process. The plate body P has a top side OS, which is visible in FIGS. 1A and 1B, and a non-visible underside, which is arranged opposite thereto and with which the plate body P is placed on the bone fragments K1 and K2.

The plate body P includes two fixation sections BA1 and BA2 and a bridge section BU located between the fixation sections BA1 and BA2. A fixation of the plate body P to the bone fragment K1 is to be implemented at the fixation section BA1 on the underside of the plate body P, wherein this fixation is achieved at fixation points BP1, which are associated with the fixation section BA1. The fixation points BP1 are designed as through-holes DO1 which extend between the top side OS and the underside and are used for guiding a respective bone screw (not shown in the figures) through. The through-holes DO1 are fixed in place on the fixation section BA1.

Adjoining the fixation section BA1, the plate body P has a bridge section BU for bridging a gap S. The gap S is located between the bone fragments K1 and K2. Adjoining the bridge section BU is the fixation section BA2 at which the plate body P is fixated to the bone fragment K2. The fixation section BA2 has fixation points BP2 associated therewith for this purpose. The fixation points BP2 are also designed as through-holes DO2 which extend between the top side OS and the underside and are used for guiding a respective bone screw (not shown in the figures) through.

In contrast to the fixation section BA1, the through-holes DO2 in the fixation section BA2 are introduced in island sections IA, which are separated from the fixation section BA2 by connecting each individual island section IA to the fixation section BA2 exclusively via spring segments FS1 and FS2. The spring segments FS1 and FS2 permit elastically resilient relative displacements of the respective island section IA, and thus also of the associated fixation point BP2, relative to the fixation section BA2.

The spring segments FS1 and FS2 and also the respective associated island section IA are defined on the osteosynthesis plate OP via through-openings D1 and D2 in the plate body P, which are U-shaped and extend from the top side OS to the underside. The through-openings D1 and D2 are nested inside one another with the openings of their U-shape facing one another, whereby in addition to separating the respective island section IA from the fixation section BA2, the spring segments FS1 and FS2 are defined. The U-shapes of the through-openings D1 and D2 correspond to one another, resulting in courses of the spring segments FS1 and FS2 that are linear and point-symmetrical with respect to one another. For this purpose, the U-shape of each of the through-openings D1 and D2 has a respective first leg S11, S12 extending at an angle outward relative to a respective base side GS1, GS2 of the U-shape, wherein a second leg S21, S22 of the respective U-shape then extends at an angle inward relative to the respective base side GS1, GS2.

FIG. 1A shows an unloaded state of the bone K, wherein the spring segments FS1 and FS2 of the respective island section IA set an initial position of the respective island section IA, thereby resulting in a gap size SM1 of the gap S between the bone fragments K1 and K2. In contrast, FIG. 1B shows the bone K under a load B, which is indicated in FIG. 1B with an arrow and represents a typical load on the bone K. This load B in the case of the preferred example embodiment of the bone K as a mandible can be a biting force to be supported.

As a special feature, the spring segments FS1 and FS2 extend at an acute angle to a force vector KV, by which a transverse force resulting on the respective fixation point BP2 due to the load B is represented and which is indicated using an arrow in FIG. 1B for one of the fixation points BP2. In addition to these acute-angled courses—respectively indicated in FIG. 1B with dashed lines—with respect to the force vector KV, the spring segments FS1 and FS2 of the respective island section IA also extend in a divergent manner toward the bridge section BU. As a result, force is deflected at the spring segments FS1 and FS2 when the load B is introduced into the bone fragment K2, causing the island sections IA to be moved away from the bridge section BU and thus increasing a distance between the island sections IA and the bridge section BU. This results in the bone fragments K1 and K2 moving apart from one another and thus the gap S increasing to a gap size SM2.

When the load B is no longer applied, the gap S then decreases once again to the gap size SM1, in that the spring segments FS1 and FS2 of the respective island section IA induce a corresponding return displacement by elastically returning to their original shape. In general, the healing process of the bone K is promoted by the relative motion of the bone fragments K1 and K2 with respect to one another.

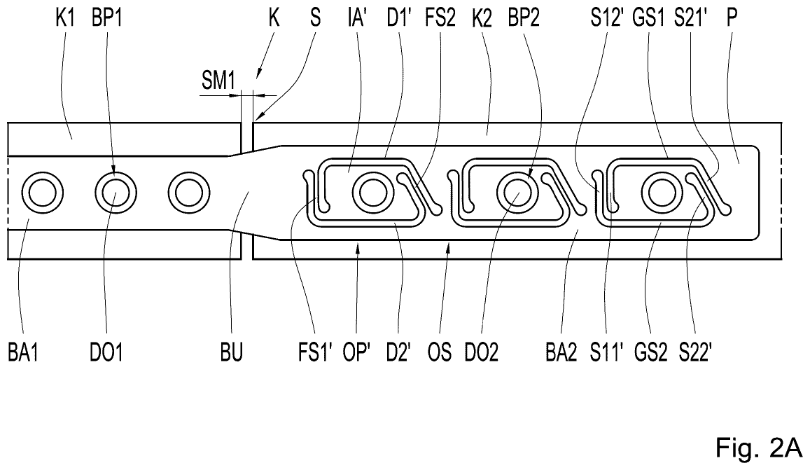

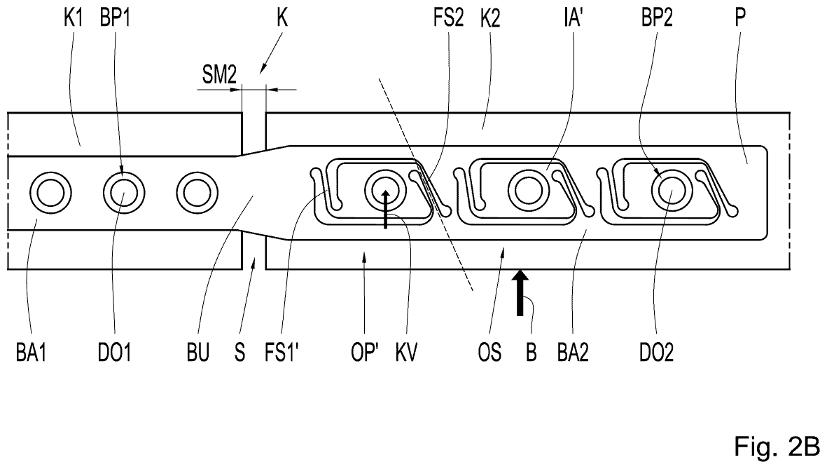

Furthermore, FIGS. 2A and 2B show schematic views of the bone K to which, in this case, an osteosynthesis plate OP′ according to a further possible example embodiment of the invention is fixated. This osteosynthesis plate OP′ largely corresponds to the osteosynthesis plate OP from FIGS. 1A and 1B, wherein, in contrast thereto, non-symmetrically designed island sections IA′ are formed on the fixation section BA2 of the plate body P in this case. This is achieved in that the respective island section IA′ and also the associated spring segments FS1′ and FS2 are formed in the present case by through-openings D1′ and D2′ which are once again U-shaped but differ from one another with respect to their respective U-shape. The two U-shapes each have a respective first leg S11′ and S12′ extending transversely to the respective base side GS1 and GS2 of the U-shape, wherein, however, in the U-shape of the through-opening D1′, a second leg S21′ extends at an angle outward relative to the base side GS1, whereas a second leg S22′ of the U-shape of the through-opening D2′ is angled inward. This yields linear and asymmetrical courses of the spring segments FS1′ and FS2 with respect to the respective fixation point BP2. Due to these asymmetrical courses, the island sections IA′ provided on the fixation section BA2 can be closely spaced.

Whereas each respective spring segment FS2 extends at an acute angle as in the example embodiment according to FIG. 1A and FIG. 1B, the asymmetrical design results in an approximately orthogonal course of the spring segment FS1′ with respect to the fixation section BA2, whereby an approximately parallel course of the spring segment FS1′ with respect to the force vector KV results in the state shown in FIG. 2B when the load B is introduced. Due to the alignment of the spring segment FS2 in relation to the force vector KV, a displacement of the island section IA′ relative to the fixation section BA2 results nevertheless, such that a distance between the island sections IA′ and the bridge section BU is increased.

For the rest, the example embodiment according to FIGS. 2A and 2B corresponds to the preceding example variant according to FIGS. 1A and 1B, such that reference is made to the descriptions thereof.

The gap sizes SM1, SM2 in FIG. 1A, FIG. 1B, FIG. 2 A and FIG. 2B and the deflection of the island sections IA in FIG. 1B and FIG. 2B are shown highly exaggerated for the sake of better illustration. In an actual application of the osteosynthesis plate OP, OP′, smaller gap sizes are used, of course, and a typical load B on the bone K would result in a smaller deflection of the island sections IA than shown in FIG. 1B and FIG. 2B.

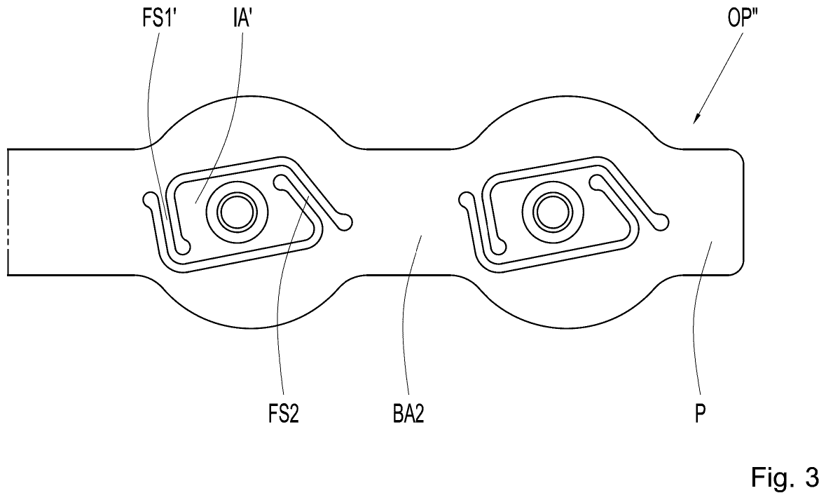

FIG. 3 shows a schematic view of a part of an osteosynthesis plate OP″ according to a further example embodiment of the invention. This example embodiment largely corresponds to the preceding example variant according to FIGS. 2A and 2B, with the difference that the island sections IA′ are then each defined as tilted about a tilt angle in the counterclockwise direction on the shown fixation section BA2 of the plate body P. Due to this tilt, each respective spring segment FS1′ and FS2 is aligned at an acute angle to the respective force vector (not shown here). The plate body P has a widened cross-section in the region of the island sections IA′. For the rest, the example embodiment according to FIG. 3 corresponds to the example variant according to FIG. 2A and FIG. 2B, such that reference is made to the descriptions thereof.

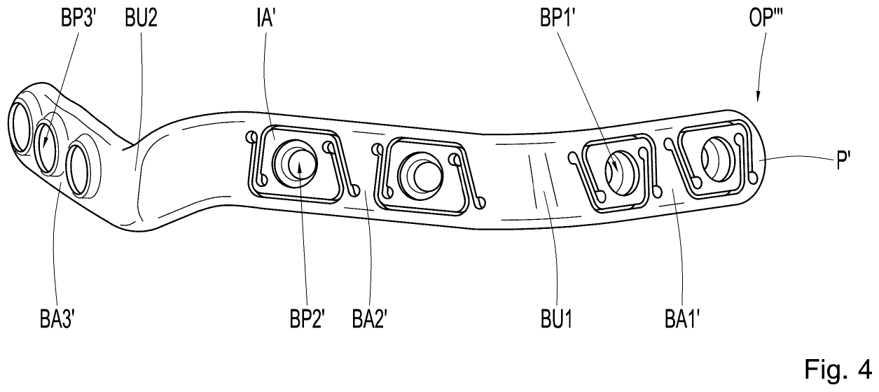

Furthermore, FIG. 4 shows a perspective representation of an osteosynthesis plate OP′″ according to a further possible example embodiment of the invention. The difference compared to the example variants described above is that a plate body P′ of this osteosynthesis plate OP′″ has two bridge sections BU1 and BU2 for bridging a respective gap (not shown in FIG. 4). Thus, three bone fragments of a bone can be stabilized with respect to one another using the osteosynthesis plate OP′″ for the restoration of said bone. The bridge sections BU1 and BU2 are defined as being interposed between fixation sections BA1′, BA2′, and BA3′, each of which is used for the fixation of a respective bone fragment. Whereas fixation points BP3′ are fixed in place on the fixation section BA3′, the fixation points BP1′ and BP2′ on the fixation sections BA1′ and BA2′ are defined on respective island sections IA′, which are designed similarly to the example variant according to FIGS. 2A and 2B. In this regard, reference is made to the descriptions of FIGS. 2A and 2B.

By the example embodiments according to example aspects of the invention, a respective osteosynthesis plate can be provided, during the use of which in the restoration of a bone, a healing process of the bone is reliably assisted.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

List of reference characters

| | |
|---|---|
| K | bone |
| OP, OP′, OP″, OP″′ | osteosynthesis plate |
| K1, K2 | bone fragments |
| P, P′ | plate body |
| OS | top side |
| BA1, BA2, BA1′, BA2′, BA3′ | fixation sections |
| BU, BU1, BU2 | bridge section |
| BP1, BP2, BP1′, BP2′, BP3′ | fixation points |
| DO1, DO2 | through-holes |
| S | gap |
| IA, IA′ | island sections |
| FS1, FS2, FS1′ | spring segments |
| D1, D2, D1′, D2′ | through-openings |
| GS1, GS2 | base sides |
| S11, S21, S11′, S21′ | leg |
| S12, S22, S12′, S22′ | leg |
| SM1, SM2 | gap sizes |
| B | load |
| KV | force vector |

1-13. (canceled)

14. An osteosynthesis plate (OP, OP′, OP″, OP′″) for fixation of bone fragments (K1, K2), comprising:
a plate body (P, P′) having a top side (OS) and an underside, the plate body (P, P′) placeable on the bone fragments (K1, K2) at the underside of the plate body (P, P′),
wherein the plate body (P, P′) comprises at least one bridge section (BU, BU1, BU2) for bridging a gap (S) between the bone fragments (K1, K2),
wherein the plate body (P, P′) comprises a plurality of fixation sections (BA1, BA2, BA1′, BA2′, BA3′) that are located on each side of the at least one bridge section (BU, BU1, BU2),
wherein a fixation point (BP1, BP2, BP1′, BP2′, BP3′) is associated with each of the fixation sections (BA1, BA2, BA1′, BA2′, BA3′), the plate body (P, P′) fixable to the bone fragments (K1, K2) at the fixation point (BP1, BP2, BP1′, BP2′, BP3′) of each of the fixation sections (BA1, BA2, BA1′, BA2′, BA3′),
wherein the fixation point (BP2, BP1′, BP2′) on one of the fixation sections (BA1, BA2, BA1′, BA2′, BA3′) is formed on an island section (IA, IA′) connected to the one of the fixation sections (BA2, BA1′, BA2′) exclusively via a plurality of spring segments (FS1, FS2, FS1′, FS2) that allow for relative displacement of the one of the fixation sections (BA2, BA1′, BA2′) and of the island section (IA, IA′) in an elastically resilient manner, and
wherein at least one of the spring segments (FS1, FS2, FS1′, FS2) is aligned in such that, when a load (B) is applied in a main loading direction of the bone (K), the island section (IA, IA′) displaces relative to the one of the fixation sections (BA2, BA1′, BA2′) and a distance between the island section (IA, IA′) and the at least one bridge section (BU, BU1) increases.

15. The osteosynthesis plate (OP, OP′, OP″, OP′″) of claim 14, wherein, when the load (B) is applied transversely to the one of the fixation sections (BA2, BA1′, BA2′), the at least one of the spring segments (FS1, FS2, FS2, FS1′, FS2) is aligned at an acute angle to a force vector (KV) that represents a transverse force resulting on the one of the fixation points (BP2, BP1′, BP2′) of the island section (IA, IA′).

16. The osteosynthesis plate (OP, OP′, OP″, OP′″) of claim 14, wherein the island section (IA, IA′) is connected to the one of the fixation sections (BA2, BA1′, BA2′) via two spring segments (FS1, FS2, FS1′, FS2).

17. The osteosynthesis plate (OP, OP′, OP″, OP′″) of claim 16, wherein the island section (IA, IA′) and the two spring segments (FS1, FS2, FS1′, FS2) are formed by two U-shaped through-openings (D1, D2, D1′, D2′), that face each other and are nested inside each other on the one of the fixation sections (BA2, BA1′, BA2′).

18. The osteosynthesis plate (OP) of claim 16, wherein the spring segments (FS1, FS2) extend point-symmetrically from the island section (IA).

19. The osteosynthesis plate (OP′, OP″, OP′″) of claim 16, wherein the spring segments (FS1′, FS2) extend asymmetrically to the plate body (P, P′).

20. The osteosynthesis plate (OP, OP′, OP″, OP′″) of claim 14, each of the fixation sections (BA2, BA1′, BA2′) is associated therewith two or more of the fixation points (BP2, BP1′, BP2′), and each of the two or more of the fixation points (BP2, BP1′, BP2′) is formed on a respective island section (IA, IA′).

21. The osteosynthesis plate (OP, OP′, OP″, OP′″) of claim 20, wherein at least one of the spring segments (FS1, FS2, FS2, FS1′, FS2) of the two or more of the fixation points (BP2, BP1′, BP2′) is aligned in such a way that the distance increases during the application of the load (B).

22. The osteosynthesis plate (OP, OP′) of claim 14, wherein:
of the fixation sections (BA1, BA2) located on each side of the at least one bridge section (BU), the at least one fixation point (BP2) on the one fixation section (BA2) is formed on the respective island section (IA, IA′), whereas the at least one fixation point (BP1) on the other fixation section (BA1) is fixed in place on the other fixation section (BA1),
the fixation point (BP2) located on a first one of the fixation sections (BA2) is formed on a respective island section (IA, IA′), and
the fixation point (BP1) on a second one of the fixation sections (BA1) is fixed on the second one of the fixation sections (BA1).

23. The osteosynthesis plate (OP′″) of claim 14, wherein each of the fixation points (BP1′, BP2) is formed on a respective island section (IA′) on each side of the at least one bridge section (BU1).

24. The osteosynthesis plate (OP′″) of claim 23, wherein at least one of the respective associated spring segments (FS1′, FS2) on the island sections (IA′) of the fixation sections (BA1′, BA2) arranged on each side of the bridge section (BU1) is aligned so as to increase the distance during the application of the load (B).

25. The osteosynthesis plate (OP, OP′, OP″) of claim 14, wherein the plate body (P) has exactly one bridge section (BU).

26. The osteosynthesis plate (OP′″) of claim 14, wherein the plate body (P′) comprises a plurality of bridge sections (BU1, BU2) for bridging a plurality of gaps (S), and respective fixation sections (BA1′, BA2′, BA3′) are provided on each side of the bridge sections (BU1, BU2).
